(19) United States
(12) Patent Application Publication
(10) Pub. No.: US 2007/0276388 A1
(43) Pub. Date: Nov. 29, 2007

(54) Surgical staples and methods of use thereof

(76) Inventors: Daniel Payne Robertson, Ocala, FL (US); David W. Szalay, Germantown, TN (US)

Correspondence Address: PEARL COHEN ZEDEK LATZER, LLP, 1500 BROADWAY 12TH FLOOR, NEW YORK, NY, 10036, US

(21) Appl. No.: 11/706,423

(22) Filed: Feb. 15, 2007

Related U.S. Application Data
(63) Continuation-in-part of application No. 11/156,457, filed on Jun. 21, 2005.
(60) Provisional application No. 60/582,081, filed on Jun. 24, 2004.

(51) Int. Cl. A61B 17/064

(52) U.S. Cl. 606/75

(57) ABSTRACT

Surgical staples and annulus closure tools, used in methods of repairing torn body tissue, implementing inter-vertebral disk surgery, sealing an inter-vertebral disk incision, preventing or inhibiting disk herniation, and/or stimulating or facilitating healing of an inter-vertebral disk; have two, three or four legs attached by connecting members. The legs optionally have projections protruding therefrom to restrict motion of the staple relative to the body tissue, and said projections may be angled to restrict motion in any desired direction.

### CROSS REFERENCE TO RELATED APPLICATIONS

This application is a Continuation-in-Part of U.S. patent application Ser. No. 11/156,457, filed Jun. 21, 2005, which claims priority of U.S. Provisional Application Ser. No. 60/582,081, filed Jun. 24, 2004. These applications are hereby incorporated in their entireties by reference herein.

### FIELD OF THE INVENTION

The present invention provides surgical staples and annulus closure tools, and methods of repairing torn body tissue, implementing inter-vertebral disk surgery, sealing an inter-vertebral disk incision, preventing or inhibiting disk herniation, and/or stimulating or facilitating healing of an inter-vertebral disk; using the surgical staples and annulus closure tools.

### BACKGROUND OF THE INVENTION

Back pain is the leading cause of healthcare expenditures in the US. The worldwide back pain market is valued at more than $2 billion and is expanding at over 25% annually. The market segment growing most rapidly is inter-vertebral disc replacement and repair, expected to exceed $1 billion by 2007.

The vertebrae of the spinal column bear the majority of the weight placed on the spine, and additionally protect the spinal cord. As can be seen with reference to FIGS. 1 and 2, inter-vertebral discs are flat, round cushions that lie between each vertebrae and absorb shock and other types of stresses. The discs have numerous functions, including maintaining separation between adjacent vertebral bodies and facilitating motion.

Each inter-vertebral disc is comprised of two main elements: a strong outer ring called the annulus fibrosus, and a soft center called the nucleus pulposus. The disc annulus is a complex structure of highly organized collagen fibrils and is the strongest layer of the disc. The disc annulus surrounds the inner nucleus pulposus, which has a more random collagen organization and proteoglycans.

FIG. 3 depicts a number of disc ailments that commonly occur. Degenerative discs are characterized by circumferential tears or fissures in the outer annulus. Bulging disks and herniated disks are characterized by localized distension of the annulus. Thinning disks result from loss of nucleus pulposus mass.

A number of strategies are currently used to repair disc ailments. In discectomy, the wall of the annulus is opened to remove a herniated disc. In artificial nucleus replacement, the nucleus pulposus (“nucleus”) is replaced with a prosthetic nucleus. Typically, an incision is made through the disc annulus to expose the nucleus, which is then removed, and dehydrated artificial nucleus implants are inserted into the cavity.

Both discectomy and artificial nucleus replacement require an effective method for sealing the incision after surgery, in order to decrease the number of disc re-herniations, preserve disc structure and retain motion segment and stability. Unsealed incisions leave an opening in the disc, which weakens the disc structure. Re-herniation through the incised annulus occurs in a significant percentage of cases (up to 30%), with 14% requiring a second operation. Presently, spinal surgeons do not have an effective method or device to repair an annulus.

It is desirable to provide an annulus closure tools and surgical staples that provide means of repairing the aforementioned ailments that are more stable and secure than previously used.

### SUMMARY OF THE INVENTION

Embodiments of the present invention provide surgical staples and annulus closure tools, and methods of repairing torn body tissue, implementing inter-vertebral disk surgery, sealing an inter-vertebral disk incision, preventing or inhibiting disk herniation, and/or stimulating or facilitating healing of an inter-vertebral disk; using the surgical staples and annulus closure tools.

In one embodiment, the present invention provides a method of sealing an inter-vertebral disk incision, comprising the step of applying a staple/closure tool to the inter-vertebral disk incision, the staple/closure tool comprising: (a) a first leg and a second leg, each with a penetrative portion terminating at a tip; and (b) a connection member connecting the first leg to the second leg, wherein the first leg and the second leg each comprise a plurality of barb-like projections protruding therefrom, of which at least one barb-like projection tapers rearwardly toward the tip of the leg whereto the one barb-like projection is attached, thereby sealing an inter-vertebral disk incision.

In another embodiment, the present invention provides a surgical staple, comprising: (a) a first leg, a second leg, and a third leg, each with a penetrative portion terminating at a tip; (b) a connection member connecting the first leg to the second leg; and (c) a connection member connecting the second leg to the third leg.

In another embodiment, the present invention provides a surgical staple, comprising: (a) a first leg, a second leg, a third leg, and a fourth leg, each with a penetrative portion terminating at a tip; (b) a first connection member connecting the first leg to the second leg; and (c) a second connection member connecting the third leg to the fourth leg; wherein the first connection member is attached to the second connection member.

In these embodiments, some or all of the legs of the surgical staple may comprise at least one projection protruding therefrom in order to assist in anchoring the surgical staple into body tissue. “Projection” could refer to any type of projection known in the art to function in anchoring a surgical staple or suture into tissue. In one embodiment, the projection could be a protruding blunt peg. In another embodiment, the projection could be a sharp spike or barb.

In still another embodiment, the projection could be angled with respect to the body of the leg so as either to prevent the legs of the surgical staple from being pushed too far into the bodily tissue or to prevent the legs of the surgical staple from being easily pulled out of the bodily tissue. In such embodiments, the projection could be an angled protruding blunt peg or an angled sharp spike or barb. Such a projection could also be conical section protruding along the side of the leg, with the portion of the conical section that is tapered toward the side of the leg allowing motion of the leg relative to the tissue in the direction against the taper, and with the flat top portion of the conical section not allowing motion of the leg relative to the tissue in the direction of the taper, i.e., towards the flat top portion, such that the orientation of the conical section determines the direction in which movement of the leg is restricted.

In an embodiment wherein the projection is designed to prevent the legs of the surgical staple from being pushed too far into the bodily tissue, the configuration of the projection should be such that it easily passes through the tissue together with the leg of the surgical staple and then has to be pushed through the tissue. In this embodiment, the projection could be angled away from the leg in the direction of the end of the leg to which the projection is attached or could be a conical section that is tapered away from the end of the leg, i.e., where the flat top portion of the conical section faces toward the end of the leg so as to act against motion of the leg in the direction of the taper, i.e., towards the end of the leg.

In an embodiment wherein the projection is designed to prevent the legs of the surgical staple from being easily pulled out of the bodily tissue, the configuration of the projection should be such that it passes easily with the legs through the tissue in the forward direction but prevents the leg from moving in the reverse direction. In this embodiment, the projection could be angled away from the leg in the direction opposite to the end of the leg to which the projection is attached or could be a conical section that is tapered toward the end of the leg, i.e., where the flat top portion of the conical section faces away from the end of the leg so as to act against motion of the leg in the direction of the taper, i.e., away from the end of the leg.

In one embodiment, the present invention provides a method of sealing an inter-vertebral disk incision, comprising the step of applying a staple/closure tool or surgical staple to the inter-vertebral disk incision, thereby sealing an inter-vertebral disk incision.

In another embodiment, the present invention provides a method of repairing a torn region of body tissue, comprising the step of applying a surgical staple of the present invention to the torn region of body tissue, thereby repairing a torn region of body tissue.

In another embodiment, the present invention provides a method of treating a ruptured inter-vertebral disk, comprising the step of applying a surgical staple of the present invention to the ruptured inter-vertebral disk, thereby treating a ruptured inter-vertebral disk.

### BRIEF DESCRIPTION OF THE DRAWINGS

The principles and operation of the system, apparatus, and method according to the present invention may be better understood with reference to the drawings, and the following description, it being understood that these drawings are given for illustrative purposes only and are not meant to be limiting, wherein:

FIG. 1 illustrates an invertebral disc anatomy.

FIG. 2 illustrates a side view of vertebral motion.

FIG. 3 illustrates an anatomical depiction of various disk ailments.

FIG. 4 illustrates degenerative changes seen in a section of porcine inter-vertebral disc in which an incision was made in the annulus.

FIGS. 5A-B illustrate the histology of a degenerative disc model.

FIG. 6 illustrates proliferation in annular cells from an incised inter-vertebral disk, as assessed by 5-bromo-2-deoxyuridine (BrdU) staining.

FIG. 7 illustrates the experimental setup for the biomechanical testing of the annulus closure tools.

FIG. 8 illustrates an annulus closure tool that has undergone expulsion of one end of the staple from a disk in which it was inserted.

FIG. 9A illustrates a first embodiment of an annulus closure tool or surgical staple.

FIG. 9B illustrates a second embodiment of an annulus closure tool or surgical staple.

FIG. 10A illustrates a third embodiment of a surgical staple.

FIG. 10B illustrates a fourth embodiment of a surgical staple.

FIG. 11A illustrates a fifth embodiment of a surgical staple.

FIG. 11B illustrates a sixth embodiment of a surgical staple.

FIGS. 11C and 11D illustrate embodiments of connection between the anchoring elements of the embodiments shown in FIGS. 11A and 11B.

FIGS. 12A-D show side views of various configurations of the surgical staple projections.

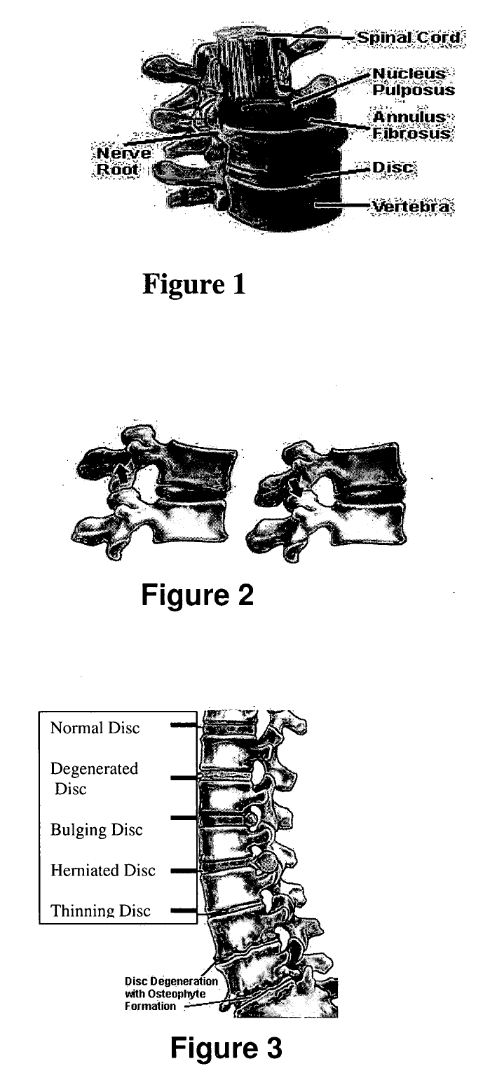

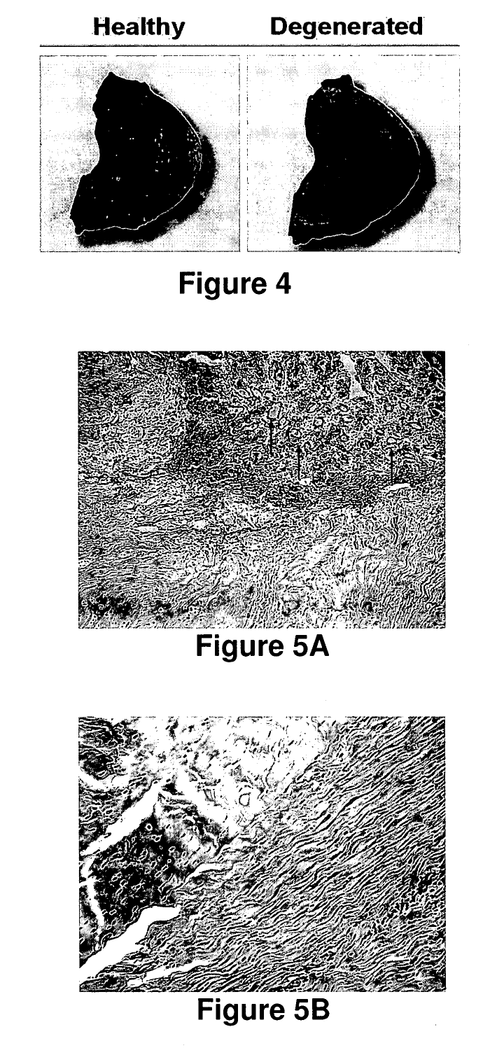

It will be appreciated that for simplicity and clarity of illustration, elements shown in the drawings have not necessarily been drawn to scale. For example, the dimensions of some of the elements may be exaggerated relative to other elements for clarity. Further, where considered appropriate, reference numerals may be repeated among the drawings to indicate corresponding or analogous elements throughout the serial views.

### DETAILED DESCRIPTION OF THE PRESENT INVENTION

Embodiments of the present invention provide surgical staples and annulus closure tools, and methods of repairing torn body tissue, implementing inter-vertebral disk surgery, sealing an inter-vertebral disk incision, preventing or inhibiting disk herniation, and/or stimulating or facilitating healing of an inter-vertebral disk; using the surgical staples and annulus closure tools.

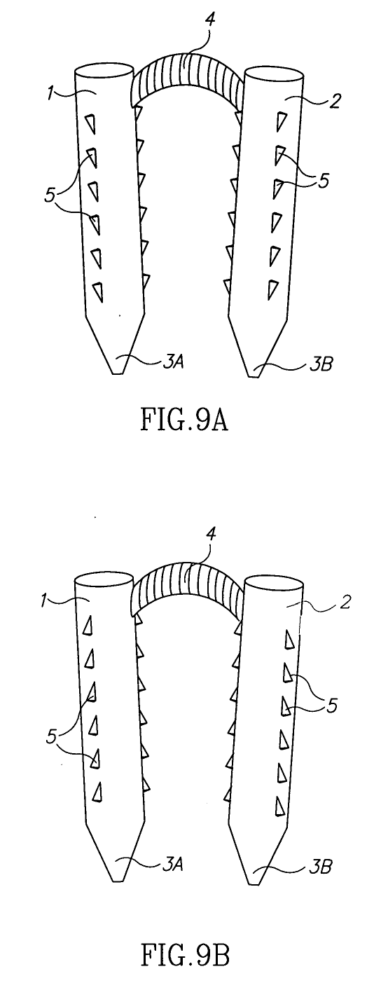

In one embodiment, as illustrated in FIG. 9A, the present invention provides an annulus closure tool or surgical staple, comprising: (a) a first anchoring member, or leg, 1 and a second anchoring member, or leg, 2, each with a body portion and a penetrative portion terminating at a tip 3; and (b) a connection member 4 connecting the first leg 1 to the second leg 2. Tips 3A,3B of legs 1,2, respectively, are tapered towards the insertion end of legs 1,2 to allow for easier insertion into bodily tissue. Either or both of the first leg 1 and the second leg 2 comprise one or more projections 5 protruding therefrom in order to assist in anchoring the tool into body tissue.

“Leg” refers, in another embodiment, to a leg of a surgical staple or annulus closure tool of the present invention. In another embodiment, the term refers to an anchoring member of a surgical staple or annulus closure tool of the present invention. Each possibility represents a separate embodiment of the present invention.

The device of the present invention is, in another embodiment, an annulus closure device. In another embodiment, the device is a surgical staple. In another embodiment, “staple” and “surgical staple” refer to a surgical staple in general. In another embodiment, the terms refer to an annulus closure device in particular. Each possibility represents a separate embodiment of the present invention.

“Projection” could refer to any type of projection known in the art to function in anchoring such a tool, e.g., surgical staple or annulus closure device, into tissue. In one embodiment, projection 5 could be a blunt peg that protrudes outward from the sides of legs 1,2. In another embodiment, projection 5 could be a sharp spike or barb. One purpose of projection 5 is to engage the bodily tissue alongside the inserted anchoring member, or leg, so as to provide stability of the staple within the bodily tissue by anchoring the staple within the bodily tissue and by restricting motion of the staple once set within the bodily tissue.

In one embodiment, projections 5 from legs 1,2 could be substantially normal, i.e., 90°, to the sides of legs 1,2 so as to equally restrict inward and outward motion of the staple relative to the bodily tissue, as shown in FIG. 12A. In another embodiment, projections 5 could be acutely angled with respect to the sides of leg 1,2 so as either to prevent the legs of the surgical staple from being pushed too far into the bodily tissue or to prevent the legs of the surgical staple from being easily pulled out of the bodily tissue, as shown in FIG. 12B. In such embodiments, projections 5 could be acutely angled with respect to the sides of legs 1, 2, with the direction of angling determining the direction of movement that is restricted.

In another embodiment, either or both of the first and second legs 1,2 comprise one or more projections that are acutely angled with respect to the body of the leg so as to restrict movement of the legs of the surgical staple in an outward direction relative to the bodily tissue. For example, as illustrated in FIG. 9A, either or both legs 1,2 comprise at least one projection that is angled away from or tapers toward the tip of the leg to which it is attached. In this embodiment, all of the projections taper toward the tip of the leg. In another embodiment, either or both legs 1,2 comprise a plurality of projections, only some of which are angled away from or taper the tip of the leg.

In this embodiment, the projection 5 is angled with respect to the body of the leg 1,2 so as to prevent the legs 1,2 of the surgical staple from being easily pulled out of the bodily tissue. In such embodiments, projection 5 could be an angled protruding blunt peg or an angled sharp spike or barb. Such a projection 5 could also resemble a conical section protruding along the side of the leg, with the side portion of the conical section being tapered toward the tip 3 of the leg and with the flat top portion of the conical section facing away from the tip 3, as shown in FIGS. 9A and 12C, thereby allowing motion of the leg into the tissue and inhibiting motion of the leg out of the tissue. In this embodiment, “tapers toward the tip of the leg” refers to a tapered configuration towards the penetration end of the legs. As a result of the tapered configuration, the projections 5 easily pass through the tissue together with the legs 1,2 in the forward direction, but are at least somewhat restricted from moving in the reverse direction. Thus, projections 5 anchor the device in the tissue and inhibit backing out of the legs from the tissue after placement. Each possibility represents a separate embodiment of the present invention.

Connection member 4 connecting first leg 1 to second leg 2 can be of any material known in the art that is suitable for connecting legs or anchoring members of surgical staples. Connection member 4 can also be attached to legs 1,2 by any means known in the art that is suitable for attaching connection members to legs or anchoring members of surgical staples.

FIG. 9B illustrates an annulus closure tool, or surgical staple, that is similar to that depicted in FIG. 9A, but wherein projections 5 are angled in the opposite direction or taper away from the tip of the leg so as to prevent the legs 1,2 of the surgical staple from being pushed too far into the bodily tissue. In this embodiment, either or both of the first and second legs 1,2 comprise one or more projections that are all angled towards the tip 3 of legs 1,2 so as to restrict movement of the legs of the surgical staple in an inward direction relative to the bodily tissue, as illustrated in FIG. 9B. In another embodiment, either or both legs 1,2 comprise a plurality of projections, only some of which are angled towards or taper away from the tip of the leg.

In this embodiment, projection 5 could be an angled protruding peg or sharp spike or barb or could also resemble a conical section protruding along the side of the leg, with the side portion of the conical section being tapered away from the tip 3 of the leg and with the flat top portion of the conical section facing the tip 3, as shown in FIGS. 9B and 12D, thereby restricting motion of the leg into the tissue. As a result of the tapered configuration, the projections 5 pass into the tissue together with the legs 1,2 in the forward direction, but are at least somewhat restricted from moving into the tissue too deeply. Thus, projections 5 anchor the device in the tissue and prevent legs 1,2 from being inserted into the tissue too far during placement.

In general, projections 4 along any of legs 1,2 may be angled in any direction desired. In another embodiment, no individual surgical staple leg comprises oppositely angled projections. Thus, in one embodiment, projections on any one leg may be angled both acutely and at 90° with respect to the leg. In an alternative embodiment, all projections on any one leg are acutely angled in the same direction. However, projections on a first leg 1 of a surgical staple or annulus closure device may not necessarily be angled in the same direction as the projections on a second leg 2.

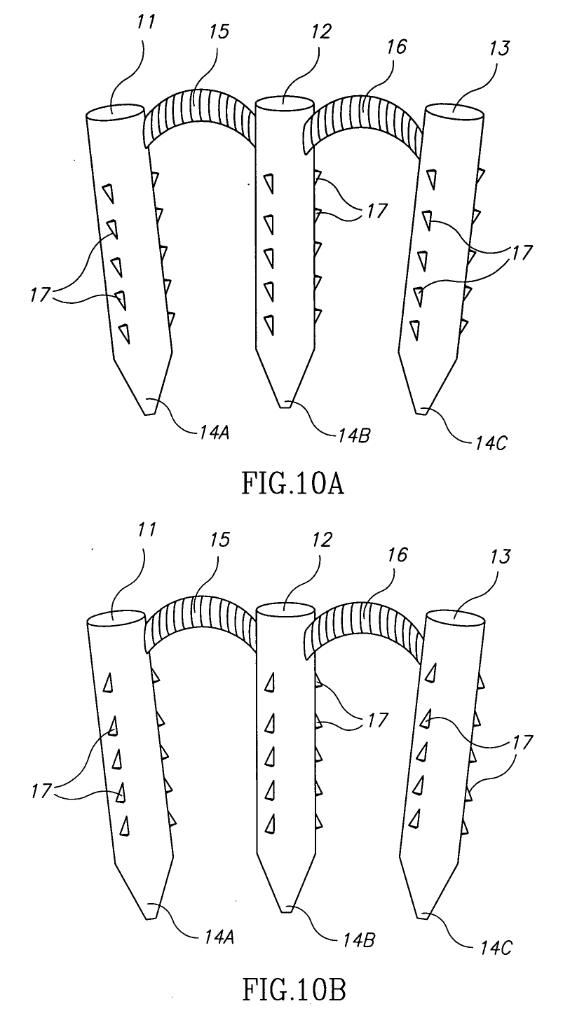

In another embodiment of the present invention, as illustrated in FIGS. 10A and 10B, the present invention provides an annulus closure tool or a surgical staple, comprising: (a) a first anchoring member, or leg 11, a second anchoring member, or leg 12, and a third anchoring member, or leg 13, each with a body portion and a penetrative portion terminating at a tip 14; (b) a connection member 15 connecting the first leg 11 to the second leg 12; and (c) a connection member 16 connecting the second leg 12 to the third leg 13. In one embodiment, legs 11,12,13 are designed to be connected using any means known in the art and inserted into the target tissue in roughly a triangular pattern. The surgical staple may also comprise a further connection member to connect first leg 11 to third leg 13 in order to more easily enable this configuration. Tips 14A,14B,14C of legs 11,12,13, respectively, are tapered, in another embodiment, towards the insertion end of legs 11,12,13 to allow for easier insertion into bodily tissue.

In one embodiment, the legs of the surgical staple or annulus closure tool of this embodiment of the present invention do not comprise projections thereon.

In an alternative embodiment, the surgical staple or annulus closure tool of the present invention comprises projections, in the various forms, configurations and angles described above. FIG. 10A shows projections 17 on legs 11,12,13 that are angled away from or taper toward the tip of the leg to which it is attached, and FIG. 10B shows projections 17 on legs 11,12,13 that are angled toward or taper away from the tip of the leg to which it is attached, each for the purposes described above. In this embodiment, all of the projections on any one leg are angled in the same direction. In another embodiment, however, the projections need not be angled in the same direction from leg to leg. In another embodiment, no individual surgical staple leg comprises oppositely angled projections, such that the projections on any one leg be angled at 90° or at less that 90° with respect to the leg.

In another embodiment, one or more of the first leg, the second leg, and the third leg of the surgical staple or annulus closure tool comprises one or more or a plurality of projections protruding therefrom. In another embodiment, two or more of the first leg, the second leg, and the third leg comprise one or more or a plurality of projections protruding therefrom. In another embodiment, each of the first leg, the second leg, and the third leg comprises one or more or a plurality of projections protruding therefrom.

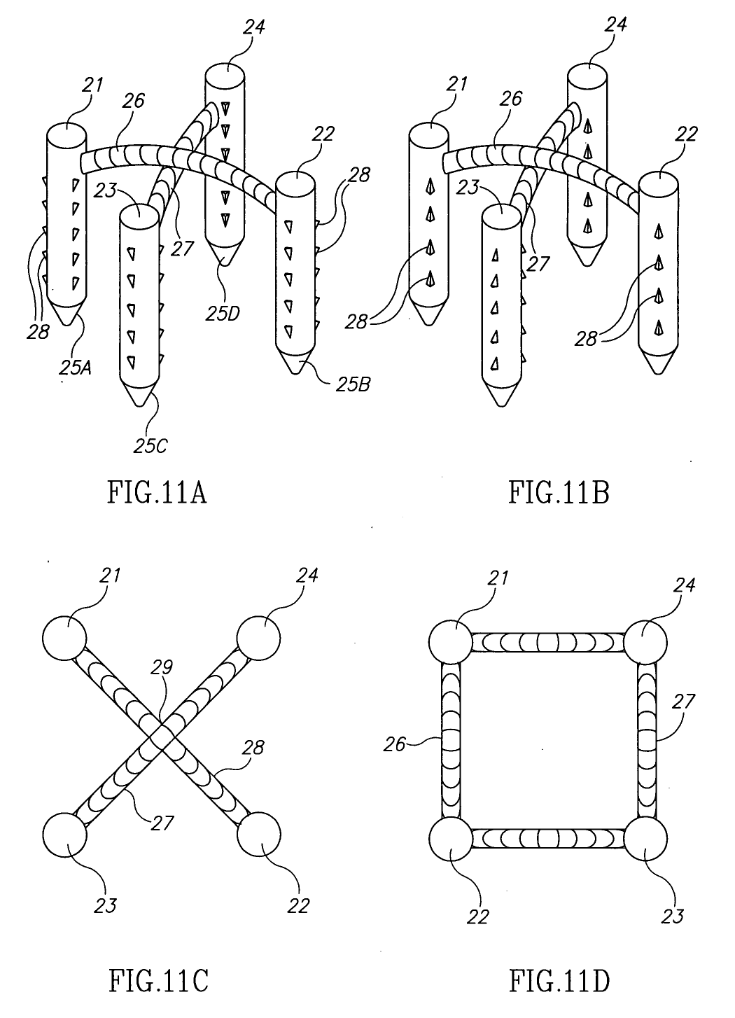

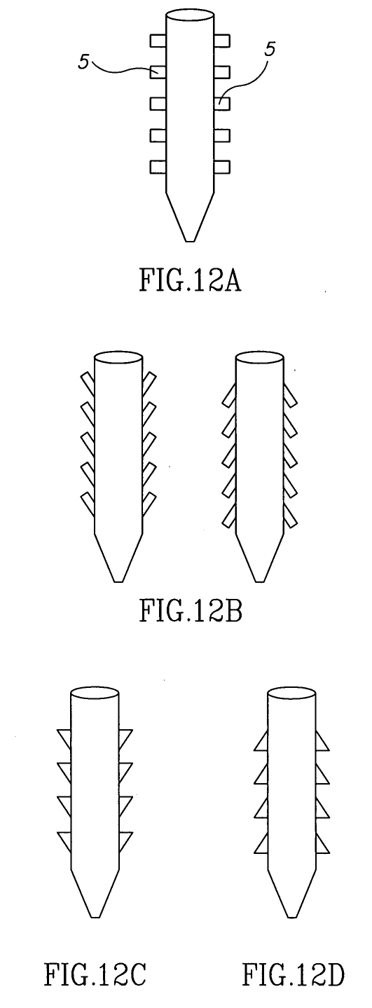

In another embodiment of the present invention, as illustrated in FIGS. 11A and 11B, the present invention provides an annulus closure tool or a surgical staple, comprising: (a) a first anchoring member, or leg 21, a second anchoring member, or leg 22, a third anchoring member, or leg 23, and a fourth anchoring member, or leg 24, each with a body portion and a penetrative portion terminating at a tip 25; (b) a first connection member 26 connecting the first leg 21 to the second leg 22; and (c) a second connection member 27 connecting the third leg 23 to the fourth leg 24. Tips 25A,25B,25C,25D of legs 21,22,23,24, respectively, are, in another embodiment, tapered towards the insertion end of legs 21,22,23,24 to allow for easier insertion into bodily tissue.

In one embodiment, legs 21,22,23,24 are designed to be inserted into the target tissue in roughly a square pattern. First connection member 26 may be attached to second connection member 27 in order using any means known in the art to more easily enable this configuration. For example, as shown in FIG. 11C, legs 21 and 22 are situated across from each other in the square pattern, and legs 23 and 24 are situated across from each other in the square pattern, such that connection members 26,27 are attached to one another at 29, which is, in another embodiment, substantially the midpoint of each of connection member 26,27, using any configuration known in the art. Alternatively, as shown in FIG. 11D, legs 21 and 22 are situated adjacent to each other in the square pattern, and legs 23 and 24 are situated adjacent to each other in the square pattern, such that there is no attachment between connection members 26,27. In addition, the configurations of connection members in FIGS. 11A and 11B can be used together.

In one embodiment, the legs of surgical staple or annulus closure tool of this embodiment of the present invention do not comprise projections thereon.

In an alternative embodiment, the surgical staple or annulus closure tool of the present invention comprises projections, in the various forms, configurations and angles described above. FIG. 11A shows projections 28 on legs 21,22,23,24 that are angled away from or taper toward the tip of the leg to which it is attached, and FIG. 11B shows projections 28 on legs 21,22,23,24 that are angled toward or taper away from the tip of the leg to which it is attached, each for the purposes described above. In this embodiment, all of the projections on any one leg are angled in the same direction. In another embodiment, however, the projections need not be angled in the same direction from leg to leg. In another embodiment, no individual surgical staple leg comprises oppositely angled projections, such that the projections on any one leg be angled at 90° or at less that 90° with respect to the leg.

In another embodiment, one or more of the first leg, the second leg, the third leg, and the fourth leg of the surgical staple or annulus closure tool comprises one or more or a plurality of projections protruding therefrom. In another embodiment, two or more of the first leg, the second leg, the third leg, and the fourth leg comprise one or more or a plurality of projections protruding therefrom. In another embodiment, each of the first leg, the second leg, the third leg, and the fourth leg comprises one or more or a plurality of projections protruding therefrom.

In another embodiment, the legs of a surgical staple of the present invention are about 5 to about 15 mm long. In another embodiment, the length of the legs is between about 6-15 mm. In another embodiment, the length is between about 7-15 mm. In another embodiment, the length is between about 8-14 mm. In another embodiment, the length is between about 9-13 mm. In another embodiment, the length is between about 10-12 mm. In another embodiment, the length is between about 10.5-11.5 mm. In another embodiment, the length is about 5-14 mm. In another embodiment, the length is about 5-13 mm. In another embodiment, the length is about 5-12 mm. In another embodiment, the length is about 5-11 mm. In another embodiment, the length is about 9-15 mm. In another embodiment, the length is about 10-15 mm. In another embodiment, the length is about 6-14 mm. In another embodiment, the length is about 7-14 mm. In another embodiment, the length is about 8-14 mm. In another embodiment, the length is about 6-13 mm. In another embodiment, the length is about 6-12 mm. In another embodiment, the length is about 6-11 mm. In another embodiment, the length is about 6-10 mm.

In another embodiment, the length of the legs is about 6 mm. In another embodiment, the length is about 7 mm. In another embodiment, the length is about 8 mm. In another embodiment, the length is about 9 mm. In another embodiment, the length is about 10 mm. In another embodiment, the length is about 11 mm. In another embodiment, the length is about 12 mm. In another embodiment, the length is about 13 mm. In another embodiment, the length is about 14 mm. In another embodiment, the length is about 15 mm. In another embodiment, the length is about 16 mm.

In another embodiment, at least two legs of a surgical staple of the present invention are of the above lengths or within one of the above ranges. (For example, at least two legs are 5-15 mm). In another embodiment, at least three legs are one of the above lengths or within one of the above ranges. In another embodiment, both legs are one of the above lengths or within one of the above ranges. In another embodiment, all three legs (of a three-legged device) are one of the above lengths or within one of the above ranges. In another embodiment, all four legs (of a four-legged device) are one of the above lengths or within one of the above ranges. Each possibility represents a separate embodiment of the present invention.

In another embodiment, at least two legs of a staple/closure tool of the present invention are one of the above lengths or within one of the above ranges. (For example, at least two legs are 5-15 mm). In another embodiment, at least three legs are one of the above lengths or within one of the above ranges. In another embodiment, both legs are one of the above lengths or within one of the above ranges. In another embodiment, all three legs (of a three-legged device) are one of the above lengths or within one of the above ranges. In another embodiment, all four legs (of a four-legged device) are one of the above lengths or within one of the above ranges. Each possibility represents a separate embodiment of the present invention.

Each length of the legs of the surgical staple and/or annulus closure tool represents a separate embodiment of the present invention.

In another embodiment, the length of a connection member of the surgical staple of the present invention is between about 5 and about 25 mm. In another embodiment, the length is between about 3-20 mm. In another embodiment, the length is between about 5-18 mm. In another embodiment, the length is between about 6-17 mm. In another embodiment, the length is between about 7-16 mm. In another embodiment, the length is between about 8-15 mm. In another embodiment, the length is between about 9-14 mm. In another embodiment, the length is between about 10-13 mm. In another embodiment, the length is between about 10.5-12.5 mm. In another embodiment, the length is between about 11-12 mm. In another embodiment, the length is about 6-25 mm. In another embodiment, the length is about 7-25 mm. In another embodiment, the length is about 8-25 mm. In another embodiment, the length is about 9-25 mm. In another embodiment, the length is about 10-25 mm. In another embodiment, the length is about 11-25 mm. In another embodiment, the length is about 12-25 mm. In another embodiment, the length is about 13-25 mm. In another embodiment, the length is about 5-24 mm. In another embodiment, the length is about 5-23 mm. In another embodiment, the length is about 5-22 mm. In another embodiment, the length is about 5-21 mm. In another embodiment, the length is about 5-20 mm. In another embodiment, the length is about 5-19 mm. In another embodiment, the length is about 5-18 mm. In another embodiment, the length is about 5-17 mm. In another embodiment, the length is about 5-16 mm. In another embodiment, the length is about 5-15 mm. In another embodiment, the length is about 5-14 mm. In another embodiment, the length is about 5-13 mm. In another embodiment, the length is about 7-24 mm. In another embodiment, the length is about 7-23 mm. In another embodiment, the length is about 7-22 mm. In another embodiment, the length is about 7-21 mm. In another embodiment, the length is about 7-20 mm. In another embodiment, the length is about 7-19 mm. In another embodiment, the length is about 7-18 mm. In another embodiment, the length is about 7-17 mm. In another embodiment, the length is about 7-16 mm. In another embodiment, the length is about 7-15 mm. In another embodiment, the length is about 7-14 mm. In another embodiment, the length is about 7-13 mm. In another embodiment, the length is about 6-20 mm. In another embodiment, the length is about 7-20 mm. In another embodiment, the length is about 8-20 mm. In another embodiment, the length is about 9-20 mm. In another embodiment, the length is about 10-20 mm. In another embodiment, the length is about 11-20 mm. In another embodiment, the length is about 12-20 mm. In another embodiment, the length is about 13-20 mm.

In another embodiment, the length of the connection member is about 6 mm. In another embodiment, the length is about 7 mm. In another embodiment, the length is about 8 mm. In another embodiment, the length is about 9 mm. In another embodiment, the length is about 10 mm. In another embodiment, the length is about 11 mm. In another embodiment, the length is about 12 mm. In another embodiment, the length is about 13 mm. In another embodiment, the length is about 14 mm. In another embodiment, the length is about 15 mm. In another embodiment, the length is about 16 mm. In another embodiment, the length is about 17 mm. In another embodiment, the length is about 18 mm. In another embodiment, the length is about 19 mm. In another embodiment, the length is about 20 mm. In another embodiment, the length is about 21 mm. In another embodiment, the length is about 22 mm. In another embodiment, the length is about 23 mm. In another embodiment, the length is about 24 mm. In another embodiment, the length is about 25 mm.

In another embodiment, the first connection member and the second connection member are each within one of the above ranges (e.g. each is within 5-25 mm long). In another embodiment, the first connection member and the second connection member are each one of the above lengths. (For example, each is about 10 mm long). Each possibility represents a separate embodiment of the present invention.

Each length of the connection member of the surgical staple and/or annulus closure tool represents a separate embodiment of the present invention.

In another embodiment, the legs of the surgical staple and/or annulus closure tool of the present invention comprise a bioresorbable material. In another embodiment, the legs are composed of a bioresorbable material. In one embodiment, the bioresorbable material is a polymer of lactide. In another embodiment, the material is a polymer of glycolide. In another embodiment, the material is a lactide/glycolide copolymer. In another embodiment, the material is poly-dioxanone. In another embodiment, the material is trimethylene carbonate. In another embodiment, the material is polyethylene oxide. In another embodiment, the material is poly(epsilon-caprolactone). In another embodiment, the material is levolactic acid. In another embodiment, the material is poly(lactic acid). In another embodiment, the material is Polylactide®. In another embodiment, the material is any other bioresorbable material known in the art that is suitable for use in a surgical staple or suture. In another embodiment, the material is a blend of two or more of the above materials. Each possibility represents a separate embodiment of the present invention.

In another embodiment, the connection member of the surgical staple and/or annulus closure tool of the present invention comprises a bioresorbable material. In another embodiment, the first connection member and the second connection member each comprises a bioresorbable material. In another embodiment, the connection member is composed of a bioresorbable material. In one embodiment, the bioresorbable material is a polymer of lactide. In another embodiment, the material is a polymer of glycolide. In another embodiment, the material is a lactide/glycolide copolymer. In another embodiment, the material is poly-dioxanone. In another embodiment, the material is trimethylene carbonate. In another embodiment, the material is polyethylene oxide. In another embodiment, the material is poly(epsilon-caprolactone). In another embodiment, the material is levolactic acid. In another embodiment, the material is poly(lactic acid). In another embodiment, the material is Polylactide®. In another embodiment, the material is any other bioresorbable material known in the art that is suitable for use in a surgical staple or suture. In another embodiment, the material is a blend of two or more of the above materials. Each possibility represents a separate embodiment of the present invention.

In another embodiment, the surgical staple and/or annulus closure tool comprises a non-bioresorbable material. In another embodiment, the surgical staple and/or annulus closure tool is composed of a non-bioresorbable material. In one embodiment, the non-bioresorbable material is nitonal. In another embodiment, the material is titanium. In another embodiment, the material is polyethylene. In another embodiment, the material is polyetheretherketone (PEEK). In another embodiment, the material is any other non-bioresorbable material known in the art that is suitable for use in a surgical staple or suture. Each possibility represents a separate embodiment of the present invention.

In another embodiment, the connection member of the surgical staple and/or annulus closure tool of methods and compositions of the present invention comprises a flexible material. In another embodiment, the first connection member and the second connection member each comprises a flexible material. In another embodiment, the connection member is composed of a flexible material. In one embodiment, the flexible material is a lactide/glycolide copolymer. In another embodiment, the flexible material is one of the above materials mentioned for inclusion in the staple/closure tool. Each material represents a separate embodiment of the present invention.

In another embodiment, the projections of the surgical staple and/or annulus closure tool of the present invention comprise a bioresorbable material. In another embodiment, the projections are composed of a bioresorbable material. In one embodiment, the bioresorbable material is a polymer of lactide. In another embodiment, the material is a polymer of glycolide. In another embodiment, the material is a lactide/glycolide copolymer. In another embodiment, the material is poly-dioxanone. In another embodiment, the material is trimethylene carbonate. In another embodiment, the material is polyethylene oxide. In another embodiment, the material is poly(epsilon-caprolactone). In another embodiment, the material is levolactic acid. In another embodiment, the material is poly(lactic acid). In another embodiment, the material is Polylactide®. In another embodiment, the material is any other bioresorbable material known in the art that is suitable for use in a surgical staple or suture. In another embodiment, the material is a blend of two or more of the above materials. Each possibility represents a separate embodiment of the present invention.

In another embodiment, the projections enable the surgical staple to be better secured in the target tissue. In another embodiment, the staple/closure tool is better secured within the inter-vertebral discs. In another embodiment, the projections or spikes protrude from the legs of the surgical staple or annulus closure tool. In another embodiment, the projections or spikes protrude from the connection member. Each possibility represents a separate embodiment of the present invention.

In one embodiment, the length of the projections is between about 0.5 to about 2.0 mm in length. In another embodiment, the length is between about 0.6 and about 1.9 mm. In another embodiment, the length is between about 0.7 to 1.8 mm. In another embodiment, the length is between about 0.8 to 1.7 mm. In one embodiment, the length is between about 0.9 to 1.6 mm. In another embodiment, the length is between about 1.0 to 1.5 mm. In another embodiment, the length is between about 1.1 to 1.4 mm. In another embodiment, the length is between about 1.15 to 1.35 mm. In another embodiment, the length is between about 1.2 to 1.25 mm.

In another embodiment, the length of the projections is about 0.6 mm. In another embodiment, the length is about 0.7 mm. In another embodiment, the length is about 0.8 mm. In another embodiment, the length is about 0.9 mm. In another embodiment, the length is about 1.0 mm. In another embodiment, the length is about 1.1 mm. In another embodiment, the length is about 1.2 mm. In another embodiment, the length is about 1.3 mm. In another embodiment, the length is about 1.4 mm. In another embodiment, the length is about 1.5 mm. Each length represents a separate embodiment of the present invention.

In one embodiment, the projections vary in length. In another embodiment, the projections have a uniform length. In another embodiment, the projections have a substantially uniform length. Each possibility represents a separate embodiment of the present invention.

In another embodiment, the projections are between about 0.5 to 2.0 mm in diameter. In another embodiment, the diameter is between about 0.6 to 1.7 mm. In another embodiment, the diameter is between about 0.7 to 1.8 mm. In another embodiment, the diameter is between about 0.8 to 1.7 mm. In one embodiment, the diameter is between about 0.9 to 1.6 mm. In another embodiment, the diameter is between about 1.0 to 1.5 mm. In one embodiment, the diameter is between about 1.1 to 1.4 mm. In another embodiment, the diameter is between about 1.15 to 1.35 mm. In another embodiment, the diameter is between about 1.2 to 1.25 mm.

In another embodiment, the diameter of the projections is about 0.6 mm. In another embodiment, the diameter is about 0.7 mm. In another embodiment, the diameter is about 0.8 mm. In another embodiment, the diameter is about 0.9 mm. In another embodiment, the diameter is about 1.0 mm. In another embodiment, the diameter is about 1.1 mm. In another embodiment, the diameter is about 1.2 mm. In another embodiment, the diameter is about 1.3 mm. In another embodiment, the diameter is about 1.4 mm. In another embodiment, the diameter is about 1.5 mm. Each diameter represents a separate embodiment of the present invention.

In one embodiment, the projections vary in diameter. In another embodiment, the projections or spikes have a uniform diameter. In another embodiment, the projections or spikes have a substantially uniform diameter. Each possibility represents a separate embodiment of the present invention.

In one embodiment, the projections protrude at an angle of between about 15 to about 90 degrees relative to the axis of the legs. In another embodiment, the angle is between about 25-80 degrees relative to the axis of the legs. In another embodiment, the angle is between about 30-75 degrees. In another embodiment, the angle is between about 35-70 degrees. In another embodiment, the angle is between about 40-65 degrees. In another embodiment, the angle is between about 45-60 degrees. In another embodiment, the angle is between about 50-55 degrees. In another embodiment, the angle is between about 35-50 degrees. In another embodiment, the angle is between about 55-70 degrees. In another embodiment, the angle is between about 60-75 degrees. In another embodiment, the angle is between about 65-80 degrees. In another embodiment, the angle is between about 70-85 degrees. In another embodiment, the angle is between about 75-90 degrees.

In another embodiment, the angle of the projections is about 30 degrees. In another embodiment, the angle is about 35 degrees. In another embodiment, the angle is about 40 degrees. In another embodiment, the angle is about 45 degrees. In another embodiment, the angle is about 50 degrees. In another embodiment, the angle is about 55 degrees. In another embodiment, the angle is about 60 degrees. In another embodiment, the angle is about 65 degrees. In another embodiment, the angle is about 70 degrees. In another embodiment, the angle is about 75 degrees. In another embodiment, the angle is about 80 degrees. In another embodiment, the angle is about 85 degrees. In another embodiment, the angle is about 90 degrees. Each angle represents a separate embodiment of the present invention.

In one embodiment, the projections vary in the angle at which they protrude relative to the axis of the legs. In another embodiment, the angle is uniform among the projections or spikes. In another embodiment, the angle is substantially uniform among the projections or spikes. Each possibility represents a separate embodiment of the present invention.

Each type of projection of the surgical staple and/or annulus closure tool represents a separate embodiment of the present invention.

In another embodiment, the connection member of the surgical staple or annulus closure tool of methods and compositions of the present invention is a convergent connection member. In another embodiment, a non-convergent connection member is utilized. In another embodiment, the convergent connection member stimulates healing of the incision by providing a convergent pressure across the incision. Each possibility represents a separate embodiment of the present invention.

In another embodiment, the connection member comprises a suture. In another embodiment, the connection member consists of a suture. Each possibility represents a separate embodiment of the present invention.

In one embodiment, a pledget is attached to or associated with a connection member of the surgical staple and/or annulus closure tool of methods and compositions of the present invention. In one embodiment, “pledget” refers to an absorbent pad. In another embodiment, the pledget is any other type of pledget known in the art. In another embodiment, the pledget is used to medicate, drain, or protect a wound or sore. In another embodiment, the pledget is flat. The use of surgical pledgets is well known in the art, and is described, for example, in U.S. Pat. Nos. 5,733,308 and 4,823,794. Each type of pledget known in the art represents a separate embodiment of the present invention.

In another embodiment, the pledget is associated with one or more of the projections, spikes, or pegs of the surgical staple and/or annulus closure tool of methods and compositions of the present invention.

In another embodiment, the pledget is associated with one or more of the legs of the surgical staple and/or annulus closure tool of methods and compositions of the present invention.

In another embodiment, the surgical staple and/or annulus closure tool of methods and compositions of the present invention further comprises a growth factor, healing factor, or healing device. The use of growth factors, healing factors, and healing devices are well known in the art, and is described, for example, in U.S. Pat. Nos. 5,703,047 and 6,810,288, and in Ichioka S et al, J Wound Care 14(3):105-9, 2005. Each type of growth factor, healing factor, and healing device known in the art represents a separate embodiment of the present invention.

In another embodiment, the pledget of methods and compositions of the present invention is impregnated with a growth factor. In another embodiment, the pledget is impregnated with a healing factor. In another embodiment, a healing device is attached to the pledget.

In another embodiment, the first leg and/or the second leg of the surgical staple and/or annulus closure tool are coated with a growth factor. In another embodiment, the first leg and/or the second leg of the surgical staple and/or annulus closure tool are coated with a healing factor. In another embodiment, the first leg and/or the second leg of the surgical staple and/or annulus closure tool are attached to a healing device.

In another embodiment, the connection member of the surgical staple and/or annulus closure tool is coated with a growth factor. In another embodiment, the connection member of the surgical staple and/or annulus closure tool is coated with a healing factor. In another embodiment, a healing device is attached to the connection member of the surgical staple and/or annulus closure tool.

In one embodiment, the surgical staple and/or annulus closure tool of methods and compositions of the present invention comprises a blunt peg at the bottom of one or more of its legs.

In another embodiment, the blunt peg is impregnated with a delivery mechanism for a growth factor or device or a healing factor or device. Each possibility represents a separate embodiment of the present invention.

In another embodiment, a device or vehicle containing a healing device, healing factor or growth factor is implanted within the surgical staple and/or annulus closure tool of methods and compositions of the present invention. In one embodiment, the device is implanted within a leg of the tool. In another embodiment, the device is implanted within the connection member of the tool. In another embodiment, the device is implanted within a spike or projection of the tool. Each possibility represents a separate embodiment of the present invention.

Each of the above types of attaching a growth factor, healing factor, or healing devices to a surgical staple and/or annulus closure tool represents a separate embodiment of the present invention.

In other embodiments, the surgical staples and annulus closure tools of any of the methods described below have any of the characteristics of a surgical staple and/or annulus closure tool of compositions of the present invention. Each characteristic represents a separate embodiment of the present invention.

In one embodiment, the present invention provides a method of sealing an inter-vertebral disk incision, comprising the step of applying a staple/closure tool to the inter-vertebral disk incision, the staple/closure tool comprising: (a) a first leg or anchoring member and a second leg or anchoring member, each with a penetrative portion terminating at a tip; and (b) a connection member connecting the first leg or anchoring member to the second leg or anchoring member, wherein the first leg or anchoring member and the second leg or anchoring member each comprise a plurality of projections protruding therefrom, of which at least one projection tapers rearwardly toward the tip of the leg or anchoring member whereto the one projection is attached, thereby sealing an inter-vertebral disk incision.

In another embodiment, the present invention provides a method of repairing a torn region of body tissue, comprising the step of applying a surgical staple of the present invention to the torn region of body tissue, thereby repairing a torn region of body tissue. In another embodiment, the legs of the staple are inserted into the target tissue in a triangular or square or in roughly a triangular or square pattern. Each possibility represents a separate embodiment of the present invention.

In another embodiment, the present invention provides a method of treating or facilitating healing of a ruptured inter-vertebral disk, comprising the step of applying a staple/closure tool of the present invention to the ruptured inter-vertebral disk, thereby treating or facilitating healing of a ruptured inter-vertebral disk. In another embodiment, the staple/closure tool is applied at a tear, fissure, or incision in the disk. In another embodiment, the legs of the closure tool are inserted into the target tissue in a triangular or square or in roughly a triangular or square pattern. Each possibility represents a separate embodiment of the present invention.

In another embodiment, the present invention provides a method of preventing or inhibiting extrusion of a nucleus pulposus or of a nucleus replacement device or material from an inter-vertebral disk, comprising the step of applying a staple/closure tool of the present invention to the inter-vertebral disk, thereby preventing or inhibiting extrusion of a nucleus pulposus or of a nucleus replacement device or material from an inter-vertebral disk. In another embodiment, the inter-vertebral disk has a tear, fissure, or incision. In another embodiment, the staple/closure tool is applied at the tear, fissure, or incision. In another embodiment, the staple/closure tool is applied at a location wherein a previous herniation occurred. In another embodiment, the legs of the closure tool are inserted into the target tissue in a triangular or square or in roughly a triangular or square pattern. Each possibility represents a separate embodiment of the present invention.

In another embodiment, the present invention provides a method of repairing a bulging or herniated inter-vertebral disk, comprising the step of applying a staple/closure tool of the present invention to a site of bulging or herniation in the inter-vertebral disk, thereby repairing a bulging or herniated inter-vertebral disk. In another embodiment, the legs of the closure tool are inserted into the target tissue in a triangular or square or in roughly a triangular or square pattern. Each possibility represents a separate embodiment of the present invention.

In another embodiment, the present invention provides a method of preventing or inhibiting re-herniation of an inter-vertebral disk, comprising the step of applying a staple/closure tool of the present invention to the inter-vertebral disk, thereby preventing or inhibiting re-herniation of an inter-vertebral disk. In another embodiment, the staple/closure tool is applied at a location wherein a previous herniation occurred. Each possibility represents a separate embodiment of the present invention. In another embodiment, the legs of the closure tool are inserted into the target tissue in a triangular or square or in roughly a triangular or square pattern. Each possibility represents a separate embodiment of the present invention.

In another embodiment of the above methods, the first and second anchoring members are placed on opposite sides of the incision.

As provided herein, the results of the present invention demonstrate that the staple/closure tool of the present invention can be inserted into and remain embedded in calf and pig inter-vertebral disks following significant and prolonged mechanical stress. Further provided herein are methods of testing the methods and compositions of the present invention, as demonstrated in the Examples herein. Thus, in another embodiment, the present invention provides methods and compositions that are optimized based on the results of testing methods disclosed herein. In another embodiment, the length of the connection member and/or the legs is modified to perform minimally invasive spine surgery (MIS spine surgery). In another embodiment, the length of the connection member and or the legs remains unmodified for performing MIS spine surgery. Each possibility represents a separate embodiment of the present invention.

The disc shape and height of the pig spine is very similar to the human spine; thus, the pig spine is a good model for methods of treating incisions and fissures in the human spine. Some differences do exist in the thicknesses of the lamina and pars articularis between pig and human spines, which resulted in restricted access to the posterior spine in the experiments performed with pig spines. This, however, did not affect the ease of implantation, once the bony tissues were adequately prepared, demonstrating that methods of the present invention have utility in human spines.

Further, methods of the present invention have utility in sealing an inter-vertebral disk incision, preventing or inhibiting herniation, and stimulating or facilitating healing of an inter-vertebral disk.

In another embodiment, one of the above methods of inhibiting extrusion or re-herniation of a nucleus replacement device or material from an inter-vertebral disk is performed following disc replacement surgery. Disc replacement surgery is a known technique for treating disk ailments involving loss of nucleus pulposus mass, e.g., thinning disks, and involves introduction of a nucleus replacement device or material into the disk. Extrusion of the device or material from the disk is a post-operative complication that prevents long-term resolution of the ailment.

In another embodiment, the inter-vertebral disk incision that is healed, closed or treated by methods of the present invention is a slit incision. In another embodiment, the incision is any other type of surgical incision known in the art. In another embodiment, the incision is a non-surgical incision. Each possibility represents a separate embodiment of the present invention.

According to some embodiments of the present invention, a method of the present invention further includes, following a surgical procedure, applying the staple/closure tool or a similar tool to the annulus incision to be closed. Application of the staple/closure tool assists, in this embodiment, in preventing or inhibiting herniation of an inter-vertebral disk following a surgical procedure. In another embodiment, any combination of the steps described herein may be implemented. In another embodiment, other steps or series of steps, in addition to those described herein, are used. In some embodiments, a method of the present invention is used to stimulate or facilitate healing of an inter-vertebral disk incision.

In another embodiment, a staple/closure tool with three legs is particularly useful for healing a ragged annular tear; e.g., from a ruptured disk. In another embodiment, the insertion of the tool at more than two points in the disk enables more secure anchoring of the tissue on the two sides of rupture. Each possibility represents a separate embodiment of the present invention.

In another embodiment, a staple/closure tool with four legs is particularly useful for healing a ragged annular tear; e.g., from a ruptured disk. In another embodiment, the insertion of the tool at four points in the disk enables more secure anchoring of the tissue on the two sides of rupture. Each possibility represents a separate embodiment of the present invention.

In another embodiment, the annulus closure tool or surgical staple of the present invention is delivered or applied to the disk using a staple/closure tool delivery device. In another embodiment, the annulus closure tool or surgical staple is delivered or applied using any other method known in the art of delivering or applying a surgical staple.

Delivery devices for surgical staples are well known in the art—see, e.g. U.S. Pat. Nos. 6,446,854 and 6,312,447. In one embodiment, the delivery device is oriented at 90 degrees relative to the annulus wall. In another embodiment, the delivery device is oriented at a different angle relative to the annulus wall. In another embodiment, pressure is applied to the delivery device before firing. In another embodiment, the handle of the delivery device is modified to enhance application of the staple/closure tool or to improve ergonomics. In another embodiment, the delivery device includes a plurality of preloaded staple/closure tools. The delivery device enables, in some embodiments, the surgical staple and/or staple/closure tool to be precisely entered into a selected area of an inter-vertebral disk. In another embodiment, delivery device enables the delivery of a surgical staple and/or staple/closure tool at a selected pressure, and provides slight deflection at the incision point, resulting in convergence and closure of the incision. Each possibility represents a separate embodiment of the present invention.

In another embodiment of the above methods, at least 2 of the anchoring members are placed on opposite sides of the incision.

In one embodiment, the delivery device comprises internal “rails” to guide the application of the surgical staple and/or annulus closure tool, as described, for example, in U.S. Pat. Nos. 6,446,854 and 6,312,447. In one embodiment, the rails of the delivery device are straight and oriented parallel to the axis of the delivery device, in order to deliver the surgical staple and/or annulus closure tool at an angle parallel to the axis of the staple/closure tool delivery device. In another embodiment, the rails of the delivery device are curved or oriented at an angle other than parallel to the axis of the delivery device, in order to deliver the surgical staple and/or annulus closure tool at an angle other than parallel to the delivery device.

Each type of delivery device represents a separate embodiment of the present invention.

In another embodiment, the present invention provides a kit comprising a reagent utilized in performing a method of the present invention. In another embodiment, the present invention provides a kit comprising a composition, tool, or instrument of the present invention.

The foregoing description of the embodiments of the invention has been presented for the purposes of illustration and description. It is not intended to be exhaustive or to limit the invention to the precise form disclosed. It should be appreciated by persons skilled in the art that many modifications, variations, substitutions, changes, and equivalents are possible in light of the above teaching. It is, therefore, to be understood that the appended claims are intended to cover all such modifications and changes as fall within the true spirit of the invention.

### EXPERIMENTAL DETAILS SECTION

### Example 1

### Biomechanical Testing of Annulus Closure Tools Materials and Experimental Methods

Storage of Samples

Fresh-frozen calf spines were sealed and stored at −20° C. These specimens were potted for 2-3 hr in an aluminum-containing epoxy, which helps to heat-sink the polymerization energy away from the spine. Samples were greased with ordinary bearing grease and covered with plastic bags as they cured in the pot fixtures to minimize moisture loss.

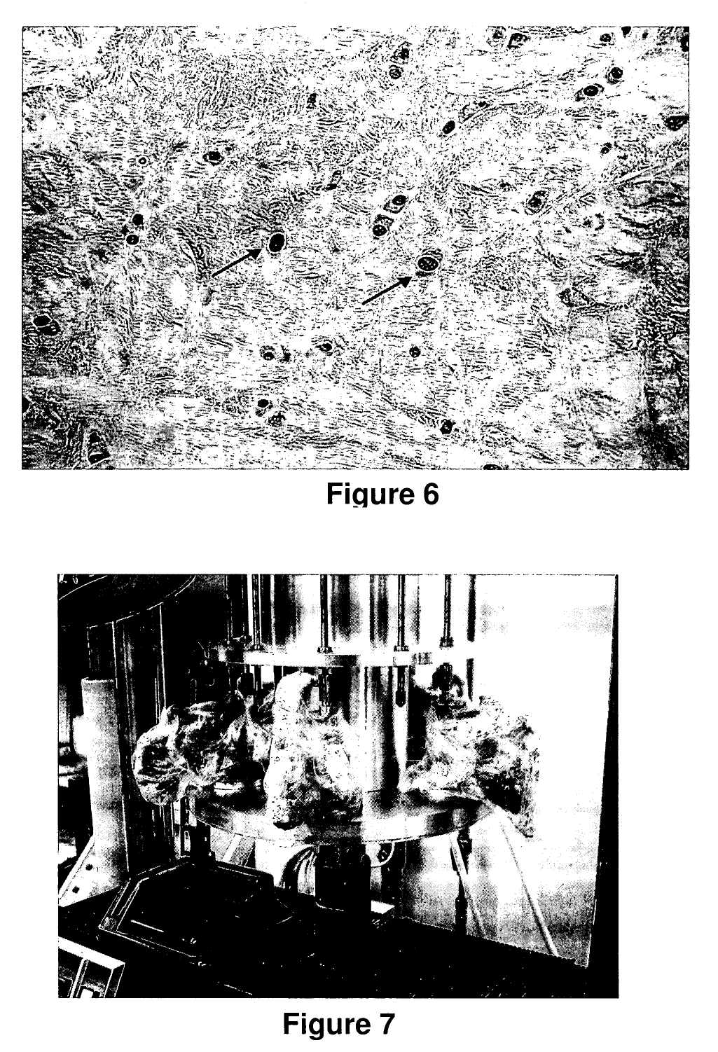

The following morning, specimens were placed in the test fixture and wrapped with Saran® wrap (FIG. 7) further trap the moisture. There was no visible change in the quality of the discs over the duration of the tests.

Never-frozen pig spines were sprayed periodically with cold saline to maintain their hydrated salt balance and temperature. The specimens warmed up to room temperature as they went through the potting process, which utilized a relatively fast-curing epoxy called marine chocking (Loctite®, Rocky Hill, Conn.). In this case, the curing process took less than 1 hr. For the low-load specimens, cure time was considerably less, as the amount of potting material was less than ⅓ of that of the high-load specimens.

### Results

Annulus closure tools of the present invention were implanted into five fresh-frozen calf spines. The closure tools were V-shaped, with legs 10 millimeter (mm) in length that had barbs protruding therefrom to anchor them in the tissue, connected by a 4 mm long braided connection member composed of a resorbable copolymer, a polyester derivative of lactic acid and glycolic acids.

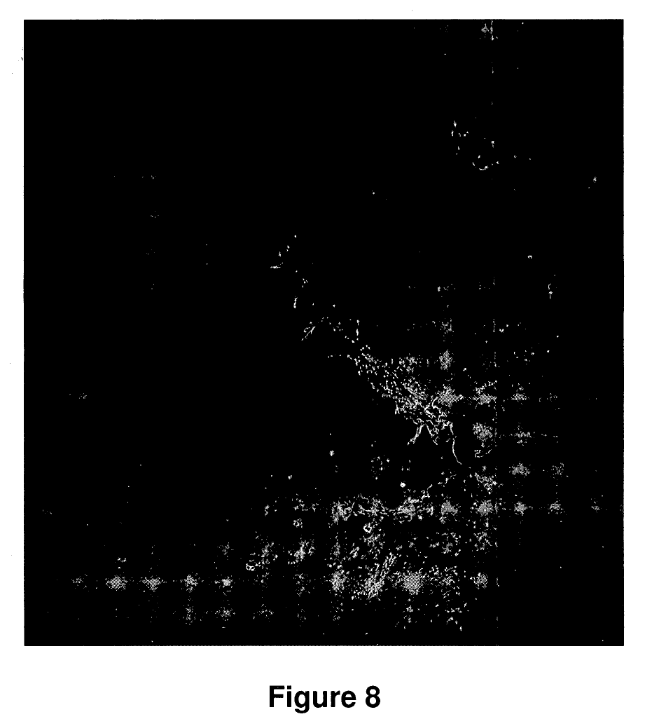

Retention of the closure tools in the first ten spines was tested using a servo-hydraulic Instron 8501 Material Testing System® (Instron Corp, Canton, Mass.). The crosshead of the test fixture was attached to the connection member, and force was applied parallel to the axis of the spine at 2 Hz, cycling between 40 and 160 pounds of force, for 34,000 cycles each over the course of 5 hours (hr). Little or no deformation of the disks was noticeable to the eye during the testing. Only one of the specimens experienced expulsion (of one end of the staple) from the disc tissues (see FIG. 8 for a representative diagram). In addition, the specimens exhibited flexion bending of 1-2 degrees during testing.

In addition, five never-frozen pig spines were tested using the above apparatus. Pigs were sacrificed on day 0, spines were harvested on day one, and testing was performed in day 2. In this case, the apparatus was run for 150,000 total cycles over 22 hr. None of these specimens showed expulsion. The tissue did not appear decayed, as extended measures were taken to assure their freshness and hydration (see Materials and Experimental Methods section). As in the previous set of specimens, these generally exhibited 1-2 degrees of flexion bending during testing.

Three additional never-frozen pig spines were subjected to high-load cyclic testing, with a significant component of flexion bending, using an electromechanical Instron 4502 Materials Testing Machine®. 5 Hz cyclic loading of between 10 and 300 pounds for about 10,000 cycles was applied to the spines over 35 min. During this time, the specimens flexed progressively, due to viscoelastic give, reaching a maximum of about 10 degrees of flexion. At this point, the minimum flexion value at the low-load end of the cycle was between 8-9 degrees. Thus, the amount of flexion induced in the specimen in each cycle did not change appreciably; rather, the offset flexion angle increased. To avoid failure of the ligamentous spine itself, the point of load application was reset slightly backwards toward the center of rotation to reduce the amount of flexion. The cyclic loading was continued for another 1.5-2 hr, totaling approximately 40,000 cycles. Only one of the three specimens exhibited expulsion (of one end of the staple) under these conditions.

The results presented in this Example demonstrate that annulus closure tools of the present invention are able to remain embedded in inter-vertebral disks following significant and prolonged mechanical stress. Thus, methods of the present invention have utility in sealing an inter-vertebral disk incision, preventing or inhibiting herniation, and stimulating or facilitating healing of an inter-vertebral disk.

What is claimed is:

1. A method of sealing an inter-vertebral disk incision or herniation, comprising the step of applying an annulus closure tool to said inter-vertebral disk incision, said annulus closure tool comprising: (a) a first anchoring member and a second anchoring member, each with a body portion and a penetrative portion terminating at a tip; and (b) a connection member connecting said first anchoring member to said second anchoring member, wherein said first anchoring member and said second anchoring member each comprise one or more projections protruding therefrom, thereby sealing an inter-vertebral disk incision or herniation.

2. The method of claim 1, wherein at least one of said one or more projections tapers toward said tip of the anchoring member to which said at least one of said one or more projections is attached.

3. The method of claim 1, wherein at least one of said one or more projections tapers away from said tip of the anchoring member to which said at least one of said one or more projections is attached.

4. The method of claim 1, wherein said inter-vertebral disk incision is a slit incision.

5. The method of claim 1, wherein said annulus closure tool comprises a bioresorbable material.

6. The method of claim 5, wherein said bioresorbable material is a polymer of lactide, glycolide, poly-dioxanone, trimethylene carbonate, polyethylene oxide, poly(epsilon-caprolactone), levolactic acid, or a blend thereof.

7. The method of claim 6, wherein said bioresorbable material is Poly(lactic acid).

8. The method of claim 1, wherein said first anchoring member and said second anchoring member are 5-15 mm long.

9. The method of claim 1, wherein said connection member is 5-25 mm long.

10. The method of claim 1, wherein said connection member comprises a flexible material.

11. The method of claim 10, wherein said flexible material is a lactide/glycolide copolymer.

12. The method of claim 1, whereby each of said one or more projections engages body tissue in the region of said inter-vertebral disk incision or herniation so as to restrict movement of said annulus closure tool with respect to said inter-vertebral disk incision or herniation.

13. A surgical staple, comprising: (a) a first anchoring member, a second anchoring member, and a third anchoring member, each with a body portion and a penetrative portion terminating at a tip; (b) a first connection member connecting said first anchoring member to said second anchoring member; and (c) a second connection member connecting said second anchoring member to said third anchoring member.

14. The surgical staple of claim 13, wherein one or more of said first anchoring member, said second anchoring member, and said third anchoring member comprises one or more projections protruding therefrom.

15. The surgical staple of claim 14, wherein at least one of said one or more projections tapers toward said tip of the anchoring member to which said at least one of said one or more projections is attached.

16. The surgical staple of claim 14, wherein at least one of said one or more projections tapers away from said tip of the anchoring member to which said at least one of said one or more projections is attached.

17. The surgical staple of claim 14, wherein each of said one or more projections engages body tissue in the region of said inter-vertebral disk incision or herniation so as to restrict movement of said annulus closure tool with respect to said inter-vertebral disk incision or herniation.

18. The surgical staple of claim 13, wherein said surgical staple comprises a bioresorbable material.

19. The surgical staple of claim 18, wherein said bioresorbable material is a polymer of lactide, glycolide, poly-dioxanone, trimethylene carbonate, polyethylene oxide, poly(epsilon-caprolactone), levolactic acid, or a blend thereof.

20. The surgical staple of claim 18, wherein said bioresorbable material is Poly(lactic acid).

21. The surgical staple of claim 13, wherein said first anchoring member, said second anchoring member, and said third anchoring member are 5-15 mm long.

22. The surgical staple of claim 13, wherein said first connection member and said second connection member are 5-25 mm long.

23. The surgical staple of claim 13, wherein said first connection member and said second connection member comprise a flexible material.

24. The surgical staple of claim 23, wherein said flexible material is a lactide/glycolide copolymer.

25. A method of repairing a torn region of body tissue, comprising the step of applying the surgical staple of claim 13 to said torn region of body tissue, thereby repairing a torn region of body tissue.

26. A method of treating a ruptured inter-vertebral disk, comprising the step of applying the surgical staple of claim 13 to said ruptured inter-vertebral disk, thereby treating a ruptured inter-vertebral disk.

27. A surgical staple, comprising: (a) a first anchoring member, a second anchoring member, a third anchoring member, and a fourth anchoring member, each with a body portion and a penetrative portion terminating at a tip; (b) a first connection member connecting said first anchoring member to said second anchoring member; and (c) a second connection member connecting said third anchoring member to said fourth anchoring member.

28. The surgical staple of claim 27, wherein one or more of said first anchoring member, said second anchoring member, said third anchoring member, and said fourth anchoring member comprises one or more projections protruding therefrom.

29. The surgical staple of claim 28, wherein said at least one of said one or more projections tapers toward said tip of the anchoring member to which said at least one of said one or more projections is attached.

30. The surgical staple of claim 28, wherein said at least one of said one or more projections tapers away from said tip of the anchoring member to which said at least one of said one or more projections is attached.

31. The surgical staple of claim 28, wherein each of said one or more projections engages body tissue in the region of said inter-vertebral disk incision or herniation so as to restrict movement of said annulus closure tool with respect to said inter-vertebral disk incision or herniation.

32. The surgical staple of claim 27, wherein said surgical staple comprises a bioresorbable material.

33. The surgical staple of claim 32, wherein said bioresorbable material is a polymer of lactide, glycolide, poly-dioxanone, trimethylene carbonate, polyethylene oxide, poly(epsilon-caprolactone), levolactic acid, or a blend thereof.

34. The surgical staple of claim 32, wherein said bioresorbable material is Poly(lactic acid).

35. The surgical staple of claim 27, wherein said first anchoring member, said second anchoring member, said third anchoring member, and said fourth anchoring member are 5-15 mm long.

36. The surgical staple of claim 27, wherein said first connection member and said second connection member are each 5-25 mm long.

37. The surgical staple of claim 27, wherein said first connection member and said second connection member each comprises a flexible material.

38. The surgical staple of claim 37, wherein said flexible material is a lactide/glycolide copolymer.

39. The surgical staple of claim 27, wherein said first connection member is attached to said second connection member.

40. A method of repairing a torn region of body tissue, comprising the step of applying the surgical staple of claim 27 to said torn region of body tissue, thereby repairing a torn region of body tissue.

41. A method of treating a ruptured inter-vertebral disk, comprising the step of applying the surgical staple of claim 27 to said ruptured inter-vertebral disk, thereby treating a ruptured inter-vertebral disk.
